(19) United States
(12) Patent Application Publication
(10) Pub. No.: US 2007/0231307 A1
(43) Pub. Date: Oct. 4, 2007

(54) Energy assisted stem cell extract

(76) Inventor: Nikolai Tankovich, San Diego, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 11/731,096

(22) Filed: Mar. 29, 2007

Related U.S. Application Data
(60) Provisional application No. 60/787,673, filed on Mar. 29, 2006.

(51) Int. Cl. A61K 35/36; A61N 5/00; A61K 31/728; A61K 31/198; A61K 38/17; A61K 38/18

(52) U.S. Cl. 424/93.7; 600/1; 424/85.1; 514/12; 514/54; 514/561

(57) ABSTRACT

A process utilizing an electromagnetic and mechanical energy to produce tissue damage in small regions of skin without damage to neighboring tissue. This permits healthy physiological responses originating in the neighboring tissue to repair the damaged tissue or to produce new healthy tissue to replace the damaged tissue including stem cells attraction. Topical application of stem cells extracts and growth factors to mobilize into the damaged area local and systemic stem cells. And a wide variety of energy sources may be utilized.

This invention relates to energy delivery devices and in particular to techniques relating to the use of energy radiation with the application of a skin regenerative composition.

### BACKGROUND OF THE INVENTION

Radiation has a wide range of applications in medicine, including aesthetic. When human tissue is damaged, natural immune and wound healing responses produce new cells to regenerate the damaged tissue. This regenerated tissue is younger and healthier than the tissue it replaced before it was damaged.

Lasers are one form of device that can be used to initiate tissue damage. In aesthetic applications, lasers have been used for hair removal, vein treatment, skin rejuvenation, treatment of telangeatesia and treatment of port wine stain. Each of these treatments is preferably preformed with a laser producing laser pulses at a wavelength chosen to be most effective for the particular treatment. For example, a Nd:YAG laser operating at 1064 nm may be used for hair removal and certain types of vein treatment. An Er:glass laser operating at 1540 nm may be used for skin rejuvenation and micro skin surgery. Treatment of port wine stain is usually performed using a dye laser operating at a wavelength of 577 nm. Lasers used for treatment of small surface veins do not work very well for treatment of larger deeper veins.

Some wavelengths are very preferentially absorbed in a particular type of tissue (referred to as a “chromophore”) that has a peak or relatively high absorption at the particular wavelength. Use of a laser beam matched to the peak or relatively high absorption in specific chromophores is referred to as “selective thermolysis”. For example the 532 nm wavelength of a frequency doubled YAG:Nd laser is highly absorbed by blood and is used to treat vascular lesions like telangeatesia and small facial veins. Some materials absorb relatively uniformly all wavelengths within broad spectral bands. Treatments directed at applying radiation energy to heat these materials are sometimes referred to as “non-selective thermolysis.” Some materials are highly reflective within specific spectral ranges which in some cases are narrow and in some cases the ranges may be very broad. For example metal particles as well as their oxides and salts tend to reflect light very well in the visible and infrared spectral ranges.

What is needed is a technique for producing damage of small sections of the skin and scalp to release inflammatory and wound healing factors, to lower the skin's barrier to penetration of compositions and components thereof topically applied to the skin, thereby creating a window into the skin for topically applied stem cells and stem cell extracts thereof to attract and mobilize skin and body stem cells to enhance a rejuvenation of epidermal structures including hair and skin.

### SUMMARY OF THE INVENTION

The present invention provides a device, composition and process for creating tissue damage as a means for enhancing the absorption by the damaged tissue of topically applied composition. In one aspect of the invention, an energy source is used to produce tissue damage in regions of the skin where the enhanced absorption of a composition is desired. For example, a laser may be used to create minor skin damage and a subsequent inflammatory response in order to release inflammatory and wound-healing factors into the skin and surrounding tissue to initiate the wound healing response. Inducing inflammation in this manner produces induration of injured tissues with interstitial fluid and a lowering of the barrier of the stratum corneum and epidermis to penetration of topically applied agents. The method includes the topical application of a composition comprising stem cell extracts that recruit local and systemic stem cells into the injured area in order to enhance the rejuvenation process.

In another aspect of the invention, a perforator, microneedle array, abrasive scrub, or other mechanical means is for initiating minor tissue injury.

In another aspect of the invention, a chemical composition, such as a peeling agent, is used as a means for initiating minor tissue injury.

The invention also concerns the formulation of a composition comprising regenerative compounds that are applied after minor tissue damage. In one aspect of the invention, the composition comprises a stem cell extract. Such a composition provides a variety of cellular factors for supplementing the natural regenerative process which includes recruiting local and systemic stem cells to the injured area. This permits healthy physiological responses originating in the neighboring tissue to repair the damaged tissue or to produce new healthy tissue to replace the damaged tissue. In preferred embodiments, a variety of radiation sources may be utilized, as well as a variety of stem cell extracts, stem cells, mobilization and differentiation compositions.

Another aspect of the invention includes a method for rejuvenating the skin comprising the steps of applying a laser to the skin of an individual to create an area of minor injury wherein the minor injury produces a local immune response, and applying a rejuvenating stem cell composition to the area of minor injury.

### BRIEF DESCRIPTION OF THE DRAWINGS

FIG. 1 is a schematic drawing of a human skin in cross section after radiation energy micro injury with inflammatory and wound healing factors release.

FIG. 2 is a drawing showing a skin with a topical stem cell extracts gel releasing stem cell components via epidermal stratum corneum opening created by radiation.

FIG. 3 shows a stem cells mobilization and wound healing factors attraction into the wide area around radiation and topical applications of stem cells extracts.

### DETAILED DESCRIPTION OF PREFERRED EMBODIMENTS

### Definitions

“Stem cell composition” refers to a composition of whole cell stem cells, a composition of stem cell extracts, or a combination of whole cell stem cells and stem cell extracts. The composition optionally comprises purified stem cell components. The stem cells for the composition may be derived from any stem cell source including embryonic stem cells, prenatal stem cells, adult stem cells, hematopoietic stem cells and fetal cord blood stem cells. One skilled in the art will appreciate that the stem cell composition may be derived from any other stem cell source known in the art at the time this application was filed.

“Minor tissue damage” or “tissue damage” refers to any damage to a treatment area that initiates a therapeutic immune response and/or the increased absorption of the rejuvenating composition through the skin barrier. Such damage produces a greater therapeutic effect than the tissue damage it causes. The damage may be induced mechanically, chemically or through an electromagnetic device such as a laser.

“Rejuvenating composition” or refers to a composition that improves the biological function of the tissue to which it is applied.

“An open skin window” or “skin window” refers to an area of skin that is subjected to damage such that the area experiences a local inflammatory response and increased permeability to topically applied compositions. The epidermal, dermal and connective tissue beneath the skin window form a stem cell microenvironment in which cells and tissues secrete growth and would healing factors that attract stem cells.

“Stem cell microenvironment” refers to the microscopic perfusions created by the tissue damage which forms a receptive area for the deposit and absorption of a composition.

### Description from Parent Patent Application

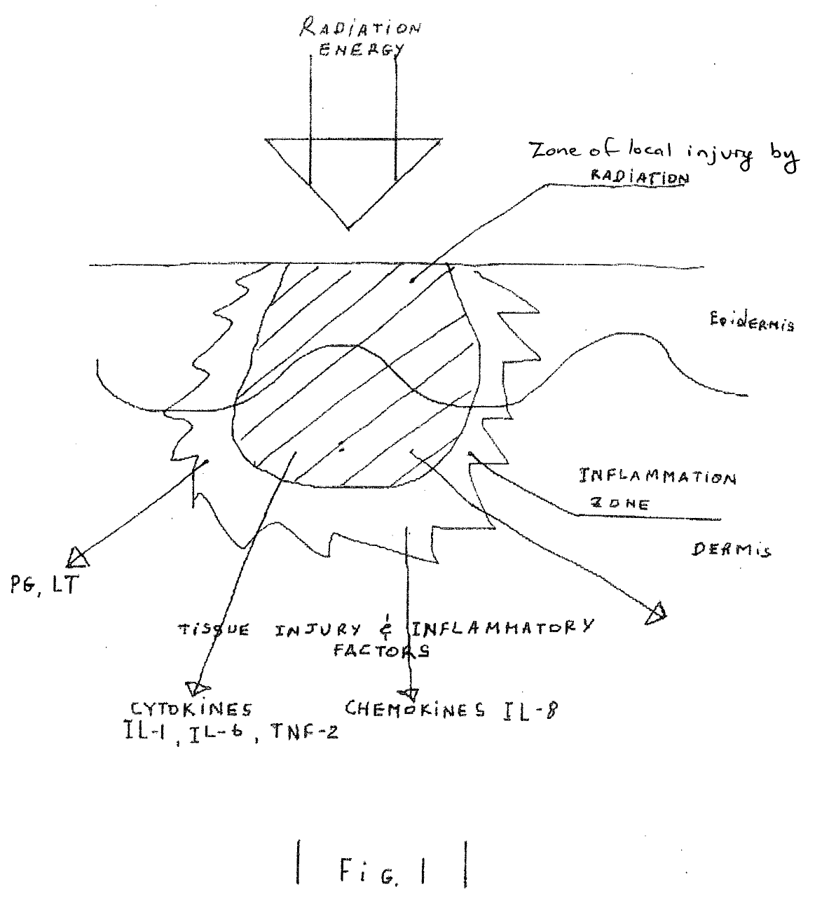

The basic concept of the present invention was described in application Ser. No. 11/229,089 filed Sep. 16, 2005 incorporated herein by reference. This application discloses the delivery of stem cells into the scalp through a skin window created by laser irradiation. FIG. 1 is a drawing from application Ser. No. 11/229,089 which includes the elements of the present invention. In particular, FIG. 1A depicts the use of electromagnetic energy to create minor damage by making microscopic holes in the skin for the deposit and penetration of a stem cell composition. Creating small holes in the skin creates a window through the stratum corneum and the epidermis for the increased absorption of regenerative compositions. In addition to providing a skin window, creating small holes in the skin induces the local release of regenerative wound healing and cell growth factors which ultimately mobilize local and systemic stem cells to the damaged area.

The effect of depositing stem cells in this manner includes accelerated wound healing and the release of growth factors and/or wound-healing factors that benefit skin rejuvenation. This is accomplished by increasing the interstitial fluid at the injury site thereby improving the diffusion of agents involved in skin growth and wound healing.

FIG. 1 is a drawing of a human skin in cross section in a region about two millimeters thick. The epidermis contains five major layers or stratums called (from top down) corneum, lucidum, granulosum, spinosum and basal. The epidermis is about 100 microns thick. Below the epidermis is the dermis that consists of two layers, the papillary and the reticular dermis. The thickness of the dermis varies from about 3 to 6 millimeters. These are the skin features that are damaged to various degrees when the skin is irradiated with any of a wide range of radiation from high intensity radiation (in the radio wave to visible light portion of the spectrum) to single photons of gamma, x-ray or ultraviolet radiation. These skin components are also the features that are rejuvenated by the inventive method and composition.

### Techniques for Leaving Healthy Skin Tissue in Radiation Treated Skin Regions

As noted above, the invention involves treating the human skin with a wide range of radiation, preferentially laser, but also including radio wave, microwave, infrared light, visible light, and ultraviolet light and even x-ray and gamma radiation. Most skin treatments using radiation produce damage to skin tissue, and the treatment relies on the patient's normal wound healing and immune processes to repair and/or replace some or all of the tissue. For example, laser hair removal produces permanent damage to some tissue but minimizes the damage to surrounding tissue. In contrast, the present invention is a device, composition and/or method for skin rejuvenation wherein the objective is to avoid permanent damage or to minimize it as much as possible. There are many available techniques for producing damage to regions while leaving healthy tissue in neighboring regions. Some of these techniques are described in the figures and in the following sections of this specification:

FIG. 1 is a drawing showing skin irradiated with a laser light creating a local minor injury. The injury of the small portion of the skin releases wound healing and immune response factors including prostaglandins (PG), leukotrienes (LT), cytokines (including IL-1, IL-6), chemokines including IL-8, tissue necrosis factors including TNF-2 as well as others. An example of electromagnetic energy radiation for medical and aesthetic use would be laser light from a 49 Watt Erbium:Glass solid state diode pump laser (available from Quantel Corporation™ with offices in Les Ulis, France; Reliant Technologies™, Inc. with offices in Mountain View, Calif.; Palomar Medical Technologies™ with offices Burlington, Mass.) emitting at a wavelength of 1.5 micron with the spot size between 50 micron to 10 mm. In creating the skin window, the number and size of laser spots depends on the level of aging and the skin's appearance. An example of electromagnetic energy radiation for home consumer use would be a 2 Watt diode laser (available from Asah Medico™ with offices in Copenhagen, Denmark) emitting at 1.4 micron with the spot size between 50 micron to 3 mm. Persons skilled in the art will be aware of many other combinations of radiation, fluences, pulse durations, spot sizes, energy levels that can be utilized for skin treatments using the concept shown in FIG. 1.

### Stem Cell Extract Composition and its Application

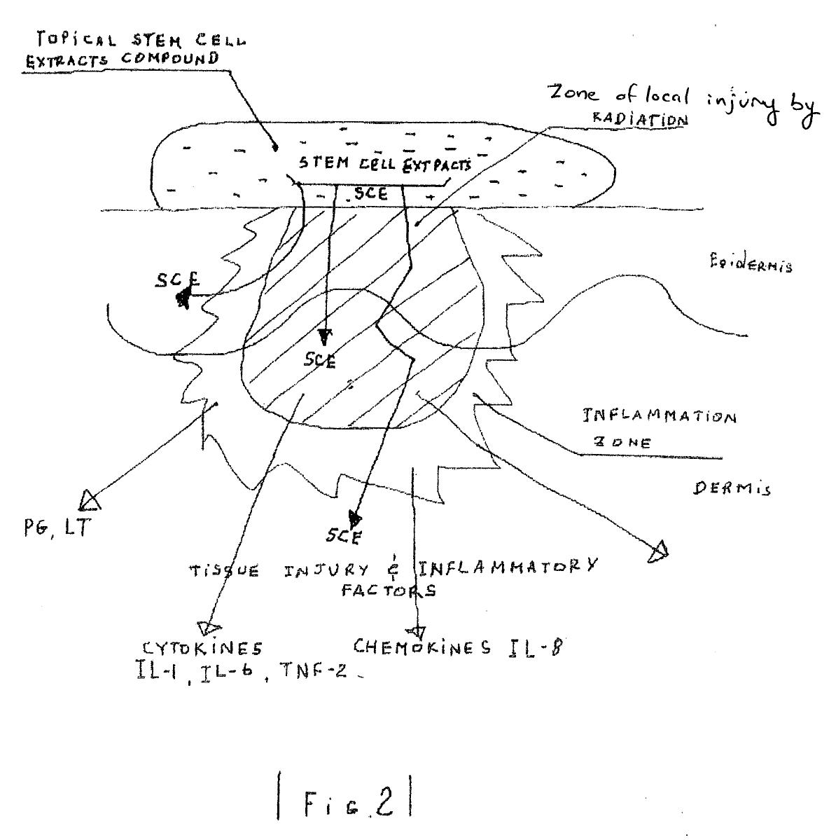

FIG. 2 is a drawing showing skin with a stem cell extract gel applied to the surface of the skin for skin rejuvenation and/or to the surface of the scalp for hair re-growth. Compositions of stem cell extracts can vary from those containing whole stem cells, to those containing complete or partial extracts of stem cells, ranging from specific genes, signaling molecules and growth factor receptors.

In applications where the regenerative composition is used for hair growth, the stem cell composition is enriched with stem cells and/or stem cell extracts from hair follicle structures including the hair papillae and/or the hair bulb.

The skin window microenvironment for attracting stem cells is enriched with transcriptional genes, expression products thereof, signaling components (including TGFβ, Notch, Wnt and Jak/Stat family members), molecules involved in telomere maintenance including telomerase, DNA methylases, transcriptional repressors of histone deacetylase, RNA helicases and the like. Enrichment of the skin window microenvironment is the result of the inflammatory response that follows minor tissue damage. Topical stem cell compositions contribute to the enrichment of the skin window environment and these compositions optionally contain non-stem cell components including tissue growth factors, hyaluronic acid, elastin, co-enzyme Q10, vitanol, D-panthenol and others. However, the percentage of each component could be any percentage from near zero to nearly one hundred percent. Different components migrate though the skin fluids with a different pace and some of them could penetrate to the blood vessels to send signals to mobilize stem cells into the damaged and surrounding tissues.

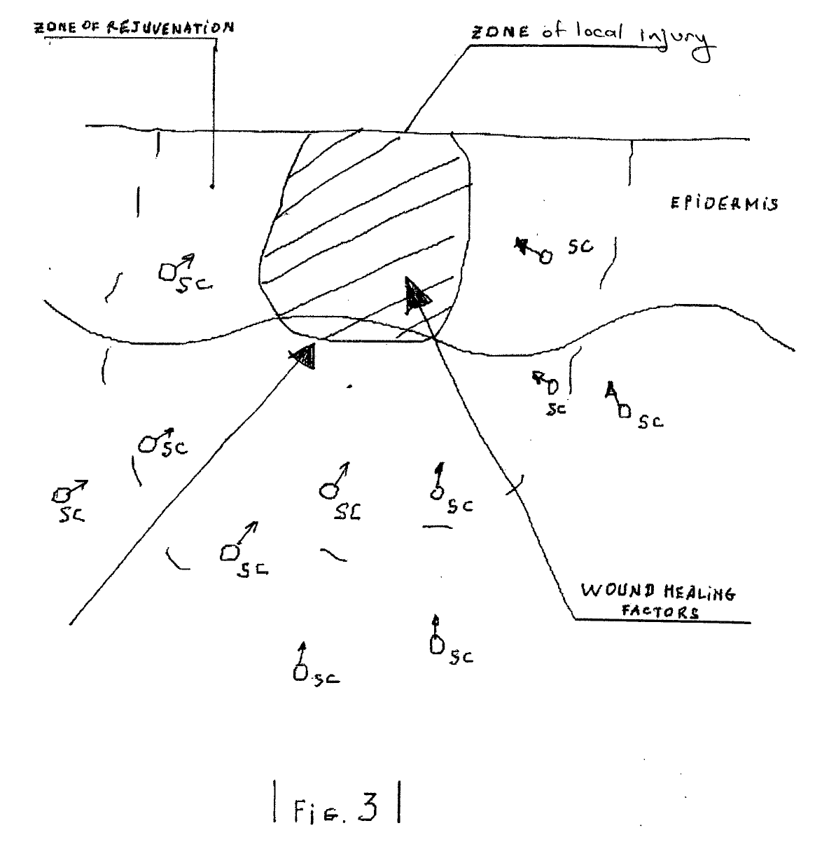

The presence and diffusion of stem cell components, growth factors, signaling molecules, wound healing and immune response factors attract stem cells locally and systemically to the injured area (see FIG. 3).

While the present invention has been described above in terms of specific preferred embodiments, persons skilled in the art will recognize that many modifications of these specific embodiments are possible without departing from the basic concepts of the present invention. Therefore, the reader should determine the scope of the present invention by the appended claims and their legal equivalents.

What is claimed is:

1. A process for skin rejuvenation comprising:
A) applying an electromagnetic or mechanical energy to produce damage to skin tissue to produce a plurality of damaged regions while protecting from damage other regions of the skin that neighbor the damaged regions;
B) allowing the persons natural wound healing and immune response factors originating in the regions of the skin to be released from the damage area to repair and/or replace skin tissue damaged in the damaged region by attracting a necessary body components including but not limited to local and systemic stem cells; and
C) applying to said skin tissue a composition comprising stem cells and/or stem cell extracts to mobilize stem cells into the damaged area.

2. The process as in claim 1 wherein the radiation source is a radiation source chosen from a group of sources consisting of the following sources: radio waves, microwaves, infrared light, visible light, and ultraviolet light and even x-ray and gamma ray radiation.

3. The process as in claim 1 wherein said stem cell extracts is a composition comprising stem cells and stem cells components.

4. The process as in claim 1 wherein said stem cell extracts composition may contain the additional non-stem cells components including growth factors and/or growth factor receptors.

5. The process as in claim 1 wherein said stem cell extracts compound further comprise one or more non-stem cell agents selected from the group consisting of tissue growth factors, hyaluronic acid, elastin, co-enzyme Q10, vitanol, D-panthenol.

6. A kit comprising:
a laser for inducing minor injury to the skin of an individual; and
a composition selected from one or both members of the group consisting of a preparation of whole stem cells, and a stem cell extract.

7. A method for enhancing uptake of a composition by the skin of an individual comprising:
irradiating the individual's skin with a laser; and
applying the composition to the irradiated skin of the individual.

8. The method of claim 8, wherein the composition comprises a stem cell composition for rejuvenating the skin of the individual.

9. A method for skin rejuvenation comprising:
A) creating a minor injury to the skin of an individual using a laser; and
B) applying a stem cell composition to the minor skin injury.

10. The method of claim 9, wherein the minor injury induces a beneficial immune response that recruits local and systemic stem cells to the minor injury.
